(19) United States
(12) Patent Application Publication
(10) Pub. No.: US 2026/0286270 A1
(43) Pub. Date: Sep. 24, 2026

(54) METHODS FOR PERFORMING PERFUSION CELL CULTURE

(71) Applicant: MERCK PATENT GMBH, Darmstadt (DE)

(72) Inventors: Jochen Bastian SIECK, Darmstadt (DE); Mona BAUSCH, Darmstadt (DE)

(73) Assignee: MERCK PATENT GMBH, Darmstadt (DE)

(21) Appl. No.: 19/475,260

(22) PCT Filed: Apr. 11, 2024
(86) PCT No.: PCT/EP2024/059797

§ 371 Date: Oct. 14, 2025

(30) Foreign Application Priority Data
Apr. 14, 2023 (EP) 23168047.1

(51) Int. Cl. C12M 1/00; C12M 1/34; C12N 5/00

(52) U.S. Cl. CPC C12M 29/10; C12M 29/26; C12M 41/32; C12N 5/0018; C12N 2500/32

(57) ABSTRACT

The present invention relates to methods and media for performing perfusion cell culture whereby a concentrated medium supplement comprising phosphotyrosine is added to the perfusion cell culture in addition to the basal medium. With this the efficiency of the cell culture can be maintained or even increased while reducing the amount of spent culture medium.

The present invention relates to methods and media for performing perfusion cell culture whereby a concentrated medium supplement is added to the perfusion cell culture in addition to the basal medium. With this the efficiency of the cell culture can be maintained or even increased while reducing the amount of spent culture medium.

The most common cultivation modes used in biomanufacturing are batch cell culture, fed-batch and perfusion cell culture. The reason for choosing one of those technologies lies in different factors linked to the protein and/or the host. Cells are cultivated either attached on carriers or in suspension. The easiest mode to operate is probably the batch bioreactor. After inoculation, cells grow and produce until a limitation due to media consumption is reached and cell density starts to decrease. The second very common process is fed-batch where nutrient limitations are prevented by adding highly concentrated feeds at different time points during the cultivation. The culture duration is therefore longer than in batch mode and volumetric productivity and space-time-yield are increased.

A perfusion cell culture process permits bioreactors to run continuously over extended periods of time up to several months by constantly perfusing fresh medium through the culture, simultaneously providing fresh nutrients for the cells and removing spent media and optionally dead cells and target product while retaining high numbers of viable cells. The key advantages of perfusion technology include higher yields per bioreactor volume, increased flexibility and more consistent product quality. But to achieve this, the system and the process need to be set up very carefully. Unlike fed-batch systems, perfusion systems accumulate no waste products. Expressed proteins can rapidly be removed and made available for purification-a significant advantage with proteins prone to instability.

Removing spent media while keeping cells in culture can be done using different technologies like filtration, e.g. alternating tangential-flow (ATF) and standard tangential-flow filtration (TFF). Other methods include use of sedimentation devices, centrifuges or an acoustic device. Another option is to retain the cells by binding them to a substrate (capillary fibers, membranes, microcarriers in fixed bed, and so on) in the bioreactor.

A review about perfusion cell culture providing details about favorable set ups can be found in “Perfusion mammalian cell culture for recombinant protein manufacturing-A critical review” Jean-Marc Bielser et al., Biotechnology Advances 36 (2018) 1328-1340. A filtration based perfusion system in which dead cells can only be removed from the system through bleeding is described in “Potential of Cell Retention Techniques for Large-Scale High-Density Perfusion Culture of Suspended Mammalian Cells”, D. Voisard, F. Meuwly, P.-A. Ruffieux, G. Baer, A. Kadouri, Cytotechnology 28:163-175, 1998.

In some perfusion processes, ultrafiltration membranes are used to retain the product in the bioreactor. Those processes are also called “concentrated fed-batch” or CFB. Concentrated fed-batch cell culture increases manufacturing capacity without additional volumetric capacity. Information about this special perfusion process can be found in William C. Yanga,*, Daniel F. Minklera, Rashmi Kshirsagarb, Thomas Ryllb, Yao-Ming Huanga, Journal of Biotechnology 217 (2016) 1-11.

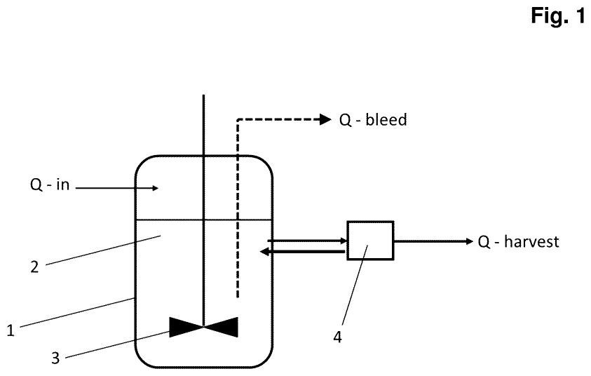

FIG. 1 shows a schematic view of a state of the art perfusion cell culture bioreactor. The bioreactor (1) with the cell culture (2) including the liquid cell culture medium and the cells is optionally stirred by stirrer 3. New, fresh medium can be added via Q-in, also called P. The harvest stream including liquid medium and target product leaves the bioreactor (1) via the Q-harvest line. Q harvest is often called H. A cell retention device (4) retains the cells e.g. by the methods described above so that cell free or cell-reduced harvest can be collected. Typically, in perfusion cell culture, media is fed continuously or semi-continuously via Q-in and harvest is removed continuously or semi-continuously via Q-harvest.

Once the cell density has reached a desired set-point, in a steady state perfusion process, excess cells are removed to keep a steady cell concentration and achieve steady-state operation. This is done via the bleed stream Q-bleed, also called B. This stream includes a liquid and a solid part, it is a suspension. The solid part includes viable and non-viable cells as well as cellular debris, the liquid part includes the liquid cell culture medium as well as waste components like cell metabolites and the target product present in the liquid. To maintain a constant volume in the bioreactor, typically Q-in =Q-harvest+Q-bleed, also called P=H+B, meaning that the volume of cell culture medium that is newly added to the bioreactor via Q-in needs to be equivalent to the volume that is removed via Q-harvest and Q-bleed.

It is also possible to run a dynamic perfusion cell culture. In this case no cells are removed, that means there is no bleed. Such a process is typically performed for a shorter time compared to steady state perfusion as described above.

The performance and yield of a process depend on the different flowrates. An increased perfusion rate generally enables the generation of more biomass and thus more target product. The faster the cells grow, the larger is the bleed rate, leading to a loss in yield. Stable operation is therefore often defined in a range where the cell density is large enough to achieve an economically viable productivity, but in a state where cell growth is controlled either by nutrient limitation or other environmental factors to minimize the bleed rate.

Consequently, in a perfusion process, cells are retained inside the bioreactor and there is typically a constant exchange of medium whereby fresh medium is provided to the cells at the same rate as the spent medium (cell waste products and medium depleted of nutrients by cell metabolism) is removed. This exchange is described as perfusion rate and expressed as vessel volumes per day (VVD) of medium exchanged.

Reducing the perfusion rate directly reduces the amount of fresh and spent medium handled by the system and may reduce strain on cell retention devices. However, higher perfusion rates can allow for higher sustained viable cell density (VCD), productivity, and quicker product removal, which may further increase product quality. The most common medium exchange rates are between 1 and 3 vvd for a cell concentration of 30 to 100 million viable cells per ml.

The medium exchange rate can also be called perfusion rate. For each cell and each perfusion process there is a specific perfusion rate needed to sustain the cells in said perfusion process. A lower perfusion rate typically results in reduced growth rate. Thus the high perfusion rates that are typically needed in perfusion cell culture are a disadvantage of this process when comparing it with e.g. batch or fed batch cell culture—the medium consumption in perfusion processes is much higher than in the other cell culture processes.

It would thus be favorable to find a way to reduce medium consumption in a perfusion cell culture process while maintaining an equivalent cell number and performance of the perfusion cell culture compared to a perfusion cell culture process only using basal medium.

It has been found that it is possible to reduce the perfusion rate of a given perfusion process by using and adding two different medium compositions instead of only using one perfusion medium. In addition to the standard basal perfusion medium which is also used to start the perfusion process a second, more concentrated medium composition, also called concentrated medium supplement, is added stepwise or gradually with increasing cell concentration/viable cell density while the perfusion rate of the standard basal perfusion medium is typically decreased.

The present invention is directed to a process for perfusion cell culture, comprising culturing cells in a bioreactor system comprising a bioreactor with a media inlet and a harvest outlet whereby
i. continuously or one or several times during the cell culture process new basal cell culture medium is inserted into the bioreactor via the media inlet
ii. continuously or one or several times during the cell culture process harvest is removed from the bioreactor via the harvest outlet
iii. one or several times during the cell culture process a concentrated medium supplement comprising at least phosphotyrosine is inserted into the bioreactor via the media inlet or an additional inlet.

In a preferred embodiment, the concentrated medium supplement comprises at least five different components.

In another preferred embodiment, the concentrated medium supplement comprising at least phosphotyrosine is inserted into the bioreactor at least 50% of the time of the cell culture, preferably at least 75% of the time.

In a very preferred embodiment, the concentrated medium supplement is inserted without increasing the overall perfusion rate. This means if cell culture concentrated medium supplement is inserted into the bioreactor with a certain perfusion rate, the perfusion rate of the basal medium is preferably at least reduced by the perfusion rate of the concentrated medium supplement.

In one embodiment, the overall perfusion rate is reduced at least once during the course of the process.

In another embodiment, the concentrated medium supplement and the basal perfusion medium are blended before addition to the bioreactor.

In a preferred embodiment, the concentrated medium supplement comprises sulfocysteine and/or 2-oxoglutaric acid and salts thereof.

In a preferred embodiment the concentration of the components of the concentrated medium supplement is at least 3 times, preferably 6 times the concentration of the equivalent component in the basal medium.

In a preferred embodiment, the overall perfusion rate (expressed mostly in vvd) calculated over the duration of the process is at least 15-50% lower compared the lowest possible perfusion rate in the same process without using the supplement. The perfusion rate can also be expressed as a cell specific medium flow rate by dividing the perfusion rate (vvd) through the viable cell density (VCD), which results in the cell specific perfusion rate (CSPR) and describes the supply of medium per cell per day. The lowest CSPR that can still maintain a steady state is described as the critical CSPR and can be determined as described in Konstantinov K, Goudar C, Ng M, Meneses R, Thrift J, Chuppa S, Matanguihan C, Michaels J, Naveh D. The “push-to-low” approach for optimization of high-density perfusion cultures of animal cells. Adv Biochem Eng Biotechnol. 2006; 101:75-98. It is important to mention that the critical CSPR is dependent on the cell line and cell culture medium combination and therefore needs to be determined for each cell line-medium combination.

In a preferred embodiment the process is initiated by inoculating the bioreactor with cells and basal cell culture medium and performing batch cell culture until perfusion is started after 2 to 5 days while the cells are still in the exponential growth phase. A suitable VCD for starting perfusion is around 4 to 6 mio cells/ml.

In a preferred embodiment concentrated medium supplement is added to the bioreactor continuously or one or several times after starting perfusion.

In another embodiment, the CSPR during perfusion, either in a steady state or a dynamic perfusion process, is reduced through the addition of the supplement in the course of the process compared to a process which only uses basal medium, whereby the performance of the two processes is comparable. For steady state perfusion processes the CSPR preferably is the critical CSPR.

In another embodiment, the CSPR, either in a steady state or a dynamic perfusion process, before entering the perfusion steady state is reduced through the addition of the supplement in the course of the process compared to a process which only uses basal medium, whereby the performance of the two processes is comparable. For steady state perfusion processes the CSPR preferably is the critical CSPR.

In a preferred embodiment, the cell specific perfusion rate of a process according to the present invention is lower compared to a perfusion process in which no concentrated medium supplement is added but which otherwise has the same or comparable VCD and/or productivity.

The present invention is also directed to a perfusion cell culture medium kit comprising a basal cell culture medium and a concentrated medium supplement comprising at least phosphotyrosine.

In a preferred embodiment the concentrated medium supplement comprises sulfocysteine and/or 2-oxoglutaric acid or salts thereof.

In a preferred embodiment the basal cell culture medium and the concentrated medium supplement are either liquid or in dry state for rehydration with a defined amount of liquid, most preferred they are in dry granulated state for rehydration with a defined amount of liquid prior to use.

FIG. 1 shows a schematic view of a state of the art perfusion bioreactor.

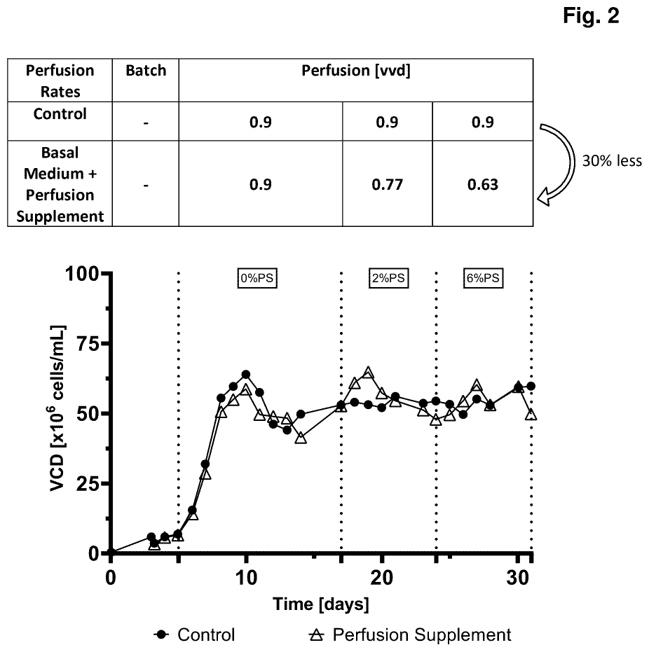

FIGS. 2 to 5 show the results of a perfusion culture experiment comparing state of the at perfusion culture with only basal medium with a perfusion culture in which the basal medium is blended with a concentrated medium supplement according to the present invention. Further details can be found in Example 1.

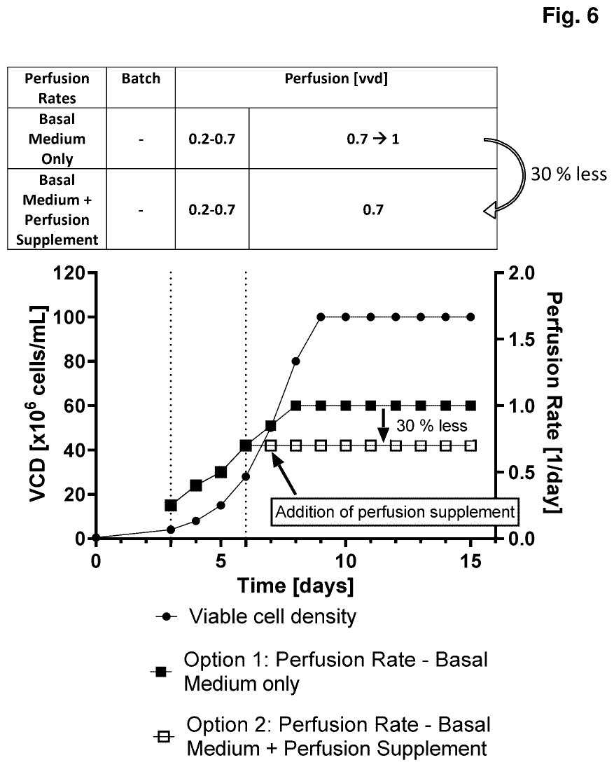

FIG. 6 shows a steady state perfusion cell culture in which a state of the art perfusion culture with only basal medium is compared with a perfusion culture in which the basal medium is blended with a concentrated medium supplement according to the present invention, whereby in this case the perfusion rate when blending the basal medium with the concentrated medium supplement is kept at a lower level compared to the state of the art perfusion culture from the beginning. Further details can be found in Example 2.

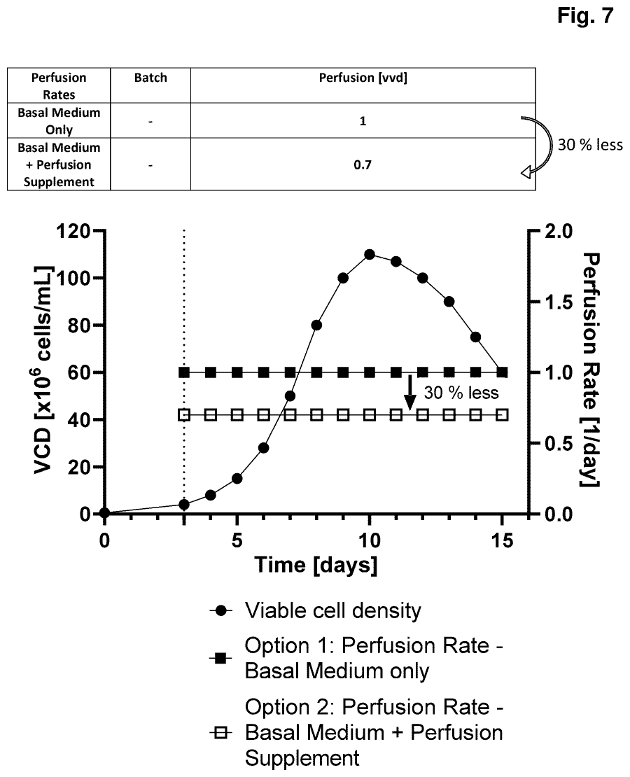

FIG. 7 shows a dynamic perfusion cell culture in which a state of the art perfusion culture with only basal medium is compared with a perfusion culture in which the basal medium is blended with a concentrated medium supplement according to the present invention, whereby the blending starts directly when starting perfusion and the perfusion rate when blending the basal medium with the concentrated medium supplement is kept at a lower level compared to the final perfusion rate of the state of the art perfusion culture. Further details can be found in Example 3.

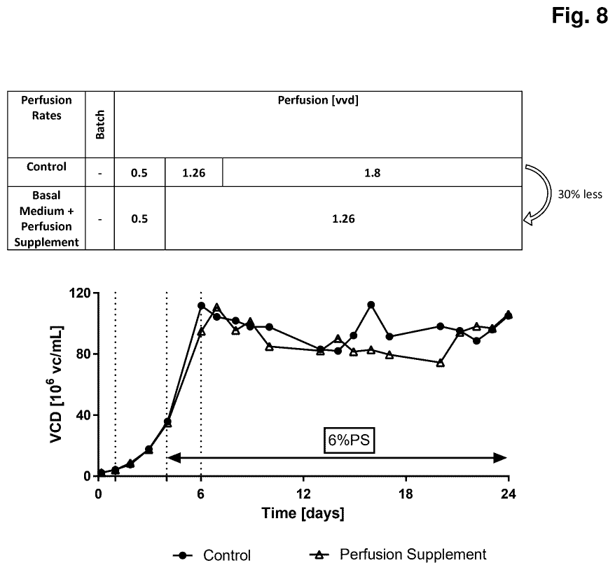

FIGS. 8 to 11 show that the perfusion supplement can be used to decrease medium demand by 30% without compromising the process performance. Further details can be found in Example 2.

A cell culture is any setup in which cells are cultured, i.e. maintained or grown.

A cell culture is typically performed in a bioreactor.

A bioreactor is any container suitable for the culture of cells, such as a bottle, tube, vessel, bag, flask and/or tank. Typically, the container is sterilized prior to use. A cell culture is typically performed by incubation of the cells in an aqueous cell culture medium under suitable conditions for growth and/or maintenance of the cells such as suitable temperature, pH, osmolality, aeration, agitation, etc. which limit contamination with foreign microorganisms from the environment. A person skilled in the art is aware of suitable incubation conditions for culturing of cells. A bioreactor used according to the present invention is preferably a bioreactor suitable for perfusion cell culture.

A bioreactor system suitable to be used in the present invention comprises the bioreactor and additional equipment that is necessary to run a perfusion cell culture in said bioreactor like one or more of the following
devices for stirring
devices for supply and discharge of components to and from the bioreactor, e.g. tubes, pumps, valves, storage tanks
a cell retention device (see above)
a system for monitoring bioreactor volume, e.g. a bioreactor balance, level sensors etc.
devices for controlling and maintaining temperature, osmolality, aeration, agitation, etc.
a computer system for automated or partially automated operation of the cell culture bioreactor

A cell culture medium (synonymously used: culture medium) according to the present invention is any mixture of components which maintains and/or supports the in vitro growth of cells and/or supports or maintains a particular physiological state.

It might comprise undefined components, such as plasma, serum, embryo extracts, or other non-defined biological extracts or peptones. It might also, preferably, be a chemically defined medium. The cell culture medium can comprise all components necessary to maintain and/or support the in vitro growth of cells or be used for the addition of selected components in combination with or not in combination with further components that are added separately (media supplement). The components of a cell culture medium are also called cell culture media ingredients.

A basal cell culture medium according to the present invention comprises all components to maintain and/or support the in vitro growth of cells and/or to support or maintain a particular physiological state in a perfusion cell culture.

A concentrated medium supplement according to the present invention comprises at least phosphotyrosine. Typically, it comprises three or more components whereby the concentration of the components is at least three times higher compared to the concentration of equivalent components in the basal cell culture medium. Typically the concentrated medium supplement comprises the components in a concentration that is between 3 and 10 times higher than the concentration of equivalent components in the basal cell culture medium. An equivalent component might be the identical chemical component or a component that can be used as a substitute for a component or a mixture of both. For example, phosphotyrosine is an equivalent to tyrosine and sulfocysteine is an equivalent to cysteine or cystine. In some cases the concentrated medium supplement cannot comprise only the identical component of the base medium as said component is not sufficiently soluble in a three to ten-fold concentrate. In this case it is favorable to fully or partially substitute said component by an equivalent component which has a higher solubility but fulfils the same function in cell culture and is suitable to substitute the component of the base medium.

A concentrated medium supplement can for example comprise one or more amino acids and/or amino acid equivalents, one or more saccharides and/or saccharide equivalents. In any case the overall concentration of the equivalent, i.e. the identical component and/or the suitable substitute, in the concentrated medium supplement is preferably at least three times, e.g. 3×, 4×, 5×, 6×, 7×, 8×, 9×, 10×, 20×, 50× higher than in the basal cell culture medium. Typically the concentration is between 3 and 50 times, preferably between 5 and 20 times higher than in the basal cell culture medium.

All concentrations refer to concentrations in the liquid medium that is added to the cell culture. Typically, the overall concentration of the components in the concentrated medium supplement is above 100 g/L, preferably, between 100 and 400 g/L, most preferred between 150 and 250 g/L.

The cell culture media and processes according to the present invention are designed to be suitable to grow or maintain/support the growth of prokaryotic cells like bacterial cells as well as eukaryotic cells like yeast, fungi, algae, plant, insect and/or mammalian cells and, optionally, archaea. Preferred cells are mammalian cells, especially CHO cells.

Chemically defined cell culture media are cell culture media comprising of chemically well characterized ‘defined’ raw materials. This means that the chemical composition of all the chemicals used in the media is known. The chemically defined media do not comprise of chemically ill-defined substances like chemically ill-defined yeast, animal or plant tissues; they do not comprise peptones, feeder cells, serum, ill-defined extracts or digests or other components which may contribute chemically poorly defined proteins and/or peptides and/or hydrolysates to the media. In some cases the chemically defined medium may comprise proteins or peptides which are chemically defined-one example is insulin (see others below).

A liquid cell culture medium is typically produced by dissolving a powdered cell culture medium in a suitable liquid.

A powdered cell culture medium or a dry powder medium or a dehydrated culture medium is a cell culture medium typically resulting from a milling process or a lyophilisation process. That means the powdered cell culture medium is typically a finely granular, particulate medium-not a liquid medium. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated. A powdered cell culture medium can also be a granulated cell culture medium, e.g. dry granulated by roller compaction or wet granulated by fluidized bed spray granulation. Such a medium can also be prepared by spray drying. Dry powder media resulting from a milling or lyophilisation process typically have particle sizes below 0.5 mm, e.g. between 0.05 and 0.5 mm. Dry powder media resulting from dry or wet granulation process, e.g. by spray drying, wet granulation or dry compaction, typically have particle sizes above 0.5 mm, e.g. between 0.5 and 5 mm.

Media which are in a dry powdered or preferably in a dry granulated state are dissolved in a suitable amount of a liquid prior to use. Concentrations of media ingredients provided herein are always directed to the concentration in the respective liquid medium whereby the skilled person is aware that dry powder media are dissolved in a certain amount of aqueous liquid to give the respective liquid medium with a certain concentration of ingredients.

Solvents, also called liquids, used to prepare a liquid cell culture medium are typically water (most particularly distilled and/or deionized water or purified water or water for injection or water purified by reverse osmosis (Milli-Q®)) or an aqueous buffer. The solvent may also comprise saline, soluble acid or base ions providing a suitable pH range (typically in the range between pH 1 and pH 10), stabilizers, surfactants, preservatives, and alcohols or other polar organic solvents.

The pH of the dissolved medium prior to addition of cells is typically between pH 2 and 12, more preferable between pH 4 and 10, even more preferably between pH 6 and 8 and most preferable between pH 6.5 to 7.5 and ideally between pH 6.8 to 7.3.

A cell culture medium which comprises all components necessary to maintain and/or support the in vitro growth of cells like the basal cell culture medium used in the present invention typically comprises at least one or more saccharide components, one or more amino acids, one or more vitamins or vitamin precursors, one or more salts, one or more buffer components, one or more co-factors and one or more nucleic acid components (nitrogenous bases) or their derivatives. It may also comprise chemically defined biochemicals such as recombinant proteins, e.g. rInsulin, rBSA, rTransferrin, rCytokines etc.

The media may also comprise sodium pyruvate, highly purified and hence chemically well-defined extracts, fatty acids and/or fatty acid derivatives and/or poloxamer product components (block copolymers based on ethylene oxide and propylene oxide) in particular Poloxamer 188 sometimes called Pluronic F 68 or Kolliphor P 188 or Lutrol F 68 and/or surface active components such as chemically prepared non-ionic surfactants. One example of a suitable non-ionic surfactants are difunctional block copolymer surfactants terminating in primary hydroxyl groups also called poloxamers, e.g. available under the trade name Pluronic® from BASF, Germany. Such poloxamer product components are in the following just called poloxamer or pluronic. Chelators, hormones and/or growth factors may also be added.

Other components it may comprise of are the pure compounds, salts, conjugates, and/or derivatives of lactic acid, thioglycollic acid, thiosulphates, tetrathionate, diaminobutane, myo-inositol, phosphatidylcholine (lecithin), sphingomyelin, iron containing compounds (including compounds with iron sulphur clusters), uric acid, carbamoyl phosphate, succinic acid, thioredoxin(s), orotic acid, phosphatidic acid, polyamines (such as putrescine, spermidine, spermine and/or cadaverine), triglycerides, steroids (including but not limited to cholesterol), metallothionine, oxygen, glycerol, urea, alpha-ketoglutarate, ammonia, glycerophosphates, starch, glycogen, glyoxylate, isoprenoids, methanol, ethanol, propanol, butanol, acetone, lipids (including but not limited to those in micelles), tributyrin, butyrin, cholic acid, desoxycholic acid, polyphosphate, acetate, tartrate, malate and/or oxalate.

Saccharide components are all mono- or di-saccharides, like glucose, galactose, ribose or fructose (examples of monosaccharides) or sucrose, lactose or maltose (examples of disaccharides) or derivatives thereof like sugar alcohols. Saccharide components may also be oligo- or polysaccharides.

Examples of amino acids according to the invention are particularly the proteinogenic amino acids, especially the essential amino acids, leucine, isoleucine, lysine, methionine, phenylalanine, threonine, tryptophan and valine, as well as the non-proteinogenic amino acids such as D-amino acids. If an amino acid is mentioned without defining if it is the D- or L-variant, both are covered, whereby the L-amino acid is preferred.

Tyrosine thus means L- or D-tyrosine, preferably L-tyrosine.

Cysteine means L- or D-cysteine, preferably L-cysteine.

The amino acid can be present as the free acid or as a metal salt thereof.

Amino acid precursors and equivalents are also included.

A suitable equivalent of tyrosine is phosphotyrosine. Phosphotyrosine means(S)-2-Amino-3-(4-phosphonooxy-phenyl)-propionic acid as well as salts thereof, like the mono-sodium salt, the di-sodium salt, the mono-potassium salt, the di-potassium salt, the calcium salt and the magnesium salt. Phosphotyrosine, also called O-Phospho-L-tyrosine has the CAS number 21820-51-9.

Suitable derivatives of cysteine are those that have been sulfonated at the SH-group of the cysteine, like(S)-2-Amino-3-sulfosulfanyl-propionic acid or salts thereof, also called sulfocysteine. The CAS number of L-Cysteine S-sulfate sodium salt sesquihydrate is 150465-29-5.

Other suitable equivalents of certain amino acids are alpha keto acids out of the group of 4-Methyl-2-oxopentanoic acid (keto Leu), 3-methyl-2-oxopentanoic acid (keto 11e), alpha-ketoisovaleric acid (keto Val), phenylpyruvic acid (keto Phe) and alpha keto gamma methylthiobutyric acid (keto Met), and/or derivatives thereof. Suitable derivatives are metal salt derivatives, peptide derivatives, like di- or tri-peptides comprising the alpha keto acid, ester derivatives as well as other derivatives, most preferred are metal salt derivatives like the sodium, potassium, calcium or magnesium salt, preferably the sodium salt.

Another suitable equivalent of amino acids are N-lactoyl-amino acids as well as salts thereof like the Na⁺, K⁺, Mg²⁺, Ca²⁺, Li⁺, preferably the Na⁺ salt thereof. A person skilled in the art is aware that either the free amino acid can be used or the H+can be substituted by a metal counterion like Na⁺ so that the salt is generated.

In a preferred embodiment, the N-lactoyl-amino acid is selected from N-lactoyl-leucine, N-lactoyl-isoleucine, N-lactoyl-valine, N-lactoyl-phenylalanine, N-lactoyl-tyrosine and/or N-lactoyl-methionine, most preferably N-lactoyl-leucine and/or N-lactoyl-isoleucine.

In a preferred embodiment the N-lactoyl amino acid is one or more of the components of formula I:

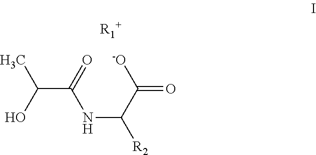

with R₁⁺ being H⁺ or a metal ion like Na⁺, K⁺, Mg²⁺, Ca²⁺, Li⁺, preferably Na⁺,
R₂ being the characteristic residue of the amino acid. In case of the amino acid being leucine, the component of formula I would be

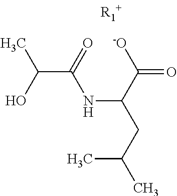

In case of the amino acid being isoleucine, the component of formula I would be

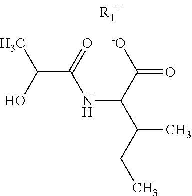

In a preferred embodiment, the equivalent is the sodium salt of the N-lactoyl amino acid. That means, preferably R₁⁺ is Na⁺.

Examples of vitamins are Vitamin A (Retinol, retinal, various retinoids, and four carotenoids), Vitamin B₁ (Thiamine), Vitamin B₂ (Riboflavin), Vitamin B₃ (Niacin, niacinamide), Vitamin B₅ (Pantothenic acid), Vitamin B₆ (Pyridoxine, pyridoxamine, pyridoxal), Vitamin B₇ (Biotin), Vitamin B₉ (Folic acid, folinic acid), Vitamin B₁₂ (Cyanocobalamin, hydroxycobalamin, methylcobalamin), Vitamin C (Ascorbic acid) (including phosphates of ascorbic acid), Vitamin D (Ergocalciferol, cholecalciferol), Vitamin E (Tocopherols, tocotrienols) and Vitamin K (phylloquinone, menaquinones). Vitamin precursors and analogues are also included.

Examples of salts are components comprising inorganic ions such as bicarbonate, calcium, chloride, magnesium, phosphate, potassium and sodium or trace elements such as Co, Cu, F, Fe, Mn, Mo, Ni, Se, Si, Ni, Bi, V and Zn. Examples are copper (II) sulphate pentahydrate (CuSO₄·5 H₂O), sodium chloride (NaCl), calcium chloride (CaCl₂·2 H₂O), potassium chloride (KCl), iron (II) sulphate, sodium phosphate monobasic anhydrous (NaH₂PO₄), magnesium sulphate anhydrous (MgSO₄), sodium phosphate dibasic anhydrous (Na₂HPO₄), magnesium chloride hexahydrate (MgCl₂·6 H₂O), zinc sulphate heptahydrate (ZnSO₄·7 H₂O).

Examples of buffers are carbonate, citrate, phosphate, HEPES, PIPES, ACES, BES, TES, MOPS and TRIS.

Examples of cofactors are compounds, salts, complexes and/or derivatives of thiamine, biotin, vitamin C, calciferol, choline, NAD/NADP (reduced and/or oxidized), cobalamin, vitamin B12, flavin mononucleotide and derivatives, flavin adenine dinucleotide and derivatives, glutathione (reduced and/or oxidized and/or as dimer), haeme, haemin, haemoglobin, ferritin, nucleotide phophates and/or derivatives (e.g. adenosine phosphates), coenzyme F420, s-adenosyl methionine, coenzyme B, coenzyme M, coenzyme Q, acetyl Co-A, molybdopterin, pyrroloquinoline quinone, tetrahydrobiopterin.

Nucleic acid components are the nucleobases, like cytosine, guanine, adenine, thymine, uracil, xanthine and/or hypoxanthine, the nucleosides like cytidine, uridine, adenosine, xanthosine, inosine, guanosine and thymidine, and the nucleotides such as adenosine monophosphate or adenosine diphosphate or adenosine triphosphate, including but not limited to the deoxy- and/or phosphate derivatives and/or dimers, trimers and/or polymers thereof, like RNA and/or DNA.

Components may be added which improve the physico-chemical properties of the media, like but not limited to, increasing clarity and/or solubility of the media and/or one or more of its components, without significantly negatively affecting the cell growth properties at the concentrations used. Such components include but are not limited to chelating agents (e.g. EDTA), antioxidants, detergents, surfactants, emulsifiers (like polysorbate 80), neutralising agents, (like polysorbate 80), micelle forming agents, micelle inhibiting agents and/or polypropylene glycol, polyethylene alcohol and/or carboxymethylcellulose.

The terms “cell density,” “viable cell density” and “cell concentration,” as used herein, refer interchangeably to the number of metabolically active cells per unit volume of a cell culture.

The terms “perfusion” or “perfusion process” refers to a cell culture process used to produce a target product, e.g., an antibody or recombinant protein, in which a high concentration of cells within a bioreactor receive fresh growth medium continually or one or more times during cell culture whereby the spent medium which may contain a target product is harvested, which means removed from the bioreactor continually or one or more times during cell culture. Preferably, fresh growth medium is continually fed into the bioreactor and spent medium which may contain the target product is harvested continually.

The exchange of medium or the amount of fresh medium fed to the bioreactor per day (perfusion rate) is expressed as vessel volumes per day (VVD) of medium exchanged. As an example, 2 L of medium being perfused daily into a system with a 2 L working volume would be expressed as 1 vvd. If not explicitly stated otherwise the perfusion rates given are the overall perfusion rates for the whole medium amount that is fed.

They might be composed of the perfusion rates of different media that are fed into the bioreactor. For example, a perfusion rate of 2 vvd might be composed of a perfusion rate of 1 vvd for the basal medium and a perfusion rate of 1 vvd for the medium concentrate.

The cells to be cultured in the system and the process of the present invention can in particular be cells capable of expressing target products, e.g. therapeutic biomolecules, such as immunoglobulins (e.g. monoclonal antibodies or antibody fragments), fusion proteins, coagulation factors, interferons, insulin, growth hormones or other recombinant proteins. Such cells can e.g. be CHO cells, Baby hamster kidney (BHK) cells, PER.C.6 cells, myeloma cells, HEK cells etc.

A “steady-state” is typically a stable condition that does not change over time or in which change in one direction is continually balanced by change in another. In perfusion, a steady state can be defined by a “constant viable cell density”. A constant viable cell density combined with a constant perfusion rate results in a constant cell-specific perfusion rate (CSPR), which is generally considered a critical criterion to achieve steady-state. As a system with living cells can of course not be kept absolutely constant, a “steady-state” is also reached if the VCD at constant perfusion rate and thus the CSPR are steady within a range of +20%, preferably around +10%. If the VCD is for example set around 50×10⁶ cells per ml, the VCD in steady state might vary between 40×10⁶ cells per ml and 60×10⁶ cells per ml.

Typically, for performing a perfusion cell culture a small number of cells and a liquid cell culture medium are introduced in the bioreactor and the cultivation conditions are selected such that the cells divide and thus produce an increasing cell density, while expressing the target product. The cultivation can be performed according to methods known in the art, involving e.g. a suitable extent of agitation, addition of oxygen/air, removal of CO₂ and other gaseous metabolites etc. During cultivation, various parameters, such as e.g. pH, conductivity, metabolite concentrations, cell density etc. can be controlled to provide suitable conditions for the given cell type. The cell density can suitably be increased to a level where the cell concentration in the bioreactor is at least 1 million cells per ml, preferably at least 10 million cells per ml, typically between 10 million and 250 million cells per ml. The upper limit will mainly be set by the rheological properties of the cell suspension at very high cell densities, where agitation and gas exchange can be hampered when paste-like consistencies are approached. Other limitations to perfusion processes might prevent the operator from achieving this physical limit, e.g. maximum cell retention device flow rate, maximum bioreactor oxygen transfer rate, limitation in product stability, and the lowest cell-specific perfusion rate (CSPR) that the medium allows. The cell viability can e.g. be at least 50%, such as at least 80% or at least 90%.

The concentration of a target product expressed by the cells in the bioreactor can be at least 0.1 g/l or at least 2 g/l. Typically it is between 0.1 and 5 g/l but in some processes like CFB, where the product is not harvested but retained in the bioreactor, product concentrations up to 10 to 30 g/l can be achieved.

An exemplary bioreactor suitable for perfusion cell culture comprises a cell retention device to keep the cells in the bioreactor during harvesting. This cell retention device can be acoustic, alternating tangential flow (ATF), a settler, a centrifuge, and the like. In some examples, disposable, reusable or semi-disposable bioreactors may be used. Any combination of hardware design may be used. In one example, a disposable cell retention device may be used. In some embodiments, disposable conduits, tubing, pumps, bag assemblies and cell retention devices are used instead of hard piping and reusable devices.

The bioreactor of the bioreactor system of the present invention may have any suitable volume including, but not limited to, about 1 L to about 2000 L, but are not limited to this exemplary range. Certain exemplary bioreactor volumes include, but are not limited to, about 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 500, 1000, 1500 L, any intermediate volumes, and the like.

An exemplary bioreactor may have any suitable minimum and maximum working volumes depending, for example, on the total vessel volume, the ratio between the height and diameter of the vessel, the vessel configuration (e.g., whether the bioreactor is a bag bioreactor), the growth rate, and the like. For example, in a 5 L bioreactor, an exemplary minimum working volume may range from about 100 mL to about 1 L, and an exemplary maximum working volume may range from about 3.5 L to about 5 L. In a 20 L bioreactor, an exemplary minimum working volume may range from about 100 mL to about 5 L, and an exemplary maximum working volume may range from about 15 L to about 19 L. In a 200 L bioreactor, an exemplary minimum working volume may range from about 20 mL to about 50 L, and an exemplary maximum working volume may range from about 150 L to about 190 L. One of ordinary skill in the art will recognize that the above numerical values and ranges are illustrative and not intended to limit the scope of the invention.

The bioreactor may include one or more inlets, also called inlet ports, for the introduction of one or more feeds (e.g., cell culture medium), chemical substances (e.g., pH buffers), anti-foam agents, and the like. It may also include one or more outlets, also called outlet ports, for the removal of cells and/or liquid from the bioreactor. Each inlet and/or outlet in the bioreactor may be provided with any suitable mechanism for initiating and conducting fluid flow through the inlet and/or outlet including, but not limited to, one or more peristaltic pumps, one or more pressurization mechanisms, and the like. Each inlet and/or outlet may be provided with any suitable mechanism for monitoring and controlling fluid flow through the inlet including, but not limited to, one or more mass flow meters, one or more flow control valves, and the like. For example, the bioreactor may include a flow control mechanism to control the flow rate of substances into and out of the bioreactor.

The bioreactor may also comprise means for volume and/or level control.

The bioreactor comprises a media inlet, that may be operated at discrete times or continuously to introduce new cell culture medium into the cell culture. The bioreactor comprises one or more harvest outlets for releasing spent cell culture, cells and/or target products. A harvest outlet may comprise a flow control valve to control the rate of harvest. In one embodiment, the harvest may be stored in a harvest bottle or container.

The bioreactor system may additionally comprise a bleed recovery device. The bleed recovery device comprises an inlet for the bleed leading the bleed from the bioreactor into the means for separating the cells of the bleed from the liquid part of the bleed and an outlet for the liquid part of the bleed leading from the means for separating the cells of the bleed from the liquid part of the bleed to the bioreactor and/or the harvest outlet. Harvest outlet in this case means any part of the harvest outlet, e.g. a tube or a harvest container. The bleed recovery device can be made of soft or hard material like metal or preferably plastic forming a defined, closed sterile volume.

A flow control valve may be provided at its inlet to control the bleed rate and the duration of the bleed extraction.

Typically the bioreactor system also comprises pumps and valves attached via the tubing. The pumps are for transport of liquids or suspensions or cell slurries from the bioreactor e.g. to the harvest or for transport of liquids or suspensions or cell slurries from e.g. the harvest or the bleed recovery device to the bioreactor or other locations. Examples of suitable pumps are peristaltic pumps, magnetically coupled pumps, membrane pumps, etc.

The valves are positioned such that they can hinder, allow or direct the flow of e.g. a liquid, cell suspension or cell slurry. Examples of suitable valves are e.g. solenoid valves or pinch valves.

The bioreactor system may include one or more sensors or probes for detecting one or more operational parameters in real-time including, but not limited to, a state of inlet ports, a state of outlet ports, a state of a multi-way manifold, a capacitance probe, a cell culture volume sensor, a cell culture bioreactor weight sensor, a liquid level sensor, a thermometer, a pH probe, an oxygen probe, a lactic acid probe, an ammonia probe, a rate of agitation sensor, a metabolic flux sensor, a metabolic rate sensor, a perfusion rate sensor, a carbon monoxide sensor, mass spectrometry, gas chromatography, combinations thereof, and the like. These sensors may detect one or more operational parameters including, but not limited to, a viable cell density (using the capacitance probe or any alternative method providing online measurements of cell density), a cell culture volume, a cell culture weight, a cell culture liquid level, a temperature, a pH, dissolved oxygen, agitation rate, metabolic flux, metabolic rate, a perfusion rate of a perfusion device, oxygen uptake rate, carbon dioxide production (e.g., using gas chromatography, mass spectrometry), lactic acid levels, ammonia levels, combinations thereof, and the like. The bioreactor may also comprise soft sensors.

The bioreactor and its inlet ports, outlet ports and the like may be coupled to one or more process management systems configured or programmed to perform multivariate analysis of sensor data and to automatically control operation of the bioreactor in real-time based on the analysis. The process management system may control operation by, for example, opening/closing a port of an inlet or an outlet, changing the state of a multi-way manifold, changing a rate of perfusion of the bioreactor system, changing a rate of agitation of the cell culture, a temperature, a pH, a level of dissolved oxygen, combinations thereof, and the like.

The present invention is directed to a process for perfusion cell culture, comprising culturing cells in a bioreactor system comprising a bioreactor with a media inlet and a harvest outlet whereby
i. continuously or one or several times during the cell culture process new cell culture medium is inserted into the bioreactor via the media inlet
ii. continuously or one or several times during the cell culture process harvest is removed from the bioreactor via the harvest outlet
iii. one or several times during the cell culture process a concentrated medium supplement comprising at least phosphotyrosine is inserted into the bioreactor via the media inlet or an additional inlet.

The inventors have found that the use of a concentrated medium supplement can advantageously be applied to perfusion cell culture without having a negative effect on the steady state of the perfusion process when using phosphotyrosine as an equivalent for tyrosine. Though phosphotyrosine cannot effectively be used in basal media for perfusion cell culture, the combination of using a concentrated media supplement and using phosphotyrosine as tyrosine equivalent in said supplement has proven especially beneficial. By adding a concentrated medium supplement comprising components which are consumed quicker by the cells than other components in the basal medium it is not necessary to add the whole basal medium for supply of those components. Instead, by adding the concentrate only those components are supplied in larger amounts while the other components are sufficiently supplied via the basal medium which can then be supplied in reduced amounts compared to a perfusion process only using basal medium.

A suitable system for performing a process according to the present invention has been described above. Perfusion is preferably realized using a cell retention device. The cell culture perfusion process can be started like known perfusion processes. This is typically done by inoculating the bioreactor with a basal medium and cells. Inoculation cell density is typically between 0.2 and 10 mio cells/ml, preferably between 0.5 and 1.0 mio cells/ml. It is also possible to start with more inoculum and thus a higher cell density.

It is possible to run the process in perfusion mode directly from the beginning, i.e. directly after inoculation with the cells. But the process is preferably first run in batch mode for some time while the number of cells, the VCD, increases. Typically, the process is run in batch mode for 2 to 8 days, preferably 3 to 5 days and then perfusion is started. In any case, perfusion is preferably turned on before growth stops being exponential. This can for example be evaluated in prior cell line characterization experiments.

Perfusion is then turned on. The final goal is to set a constant perfusion rate and a constant bleed rate to reach a constant VCD. In the process according to the present invention, perfusion can be started by feeding only basal cell culture medium or by feeding a combination of basal medium and concentrated medium supplement. Preferably, the perfusion phase is started with only feeding basal cell culture medium. The perfusion rate is preferably around 0.5 to 3 vvd, preferably between 1 and 1.5. After reaching a constant VCD, the medium composition and amount can then be altered by reducing the overall perfusion rate and enlarging the proportion of the concentrated medium supplement in the overall amount of medium. It is possible to alter the medium composition one or several times, e.g. two times, three times or four times, whereby the proportion of the concentrated medium supplement is typically enlarged and the overall perfusion rate is reduced. The overall perfusion rate is the perfusion rate of the basal medium and the medium concentrate. The bleed rate is of course adjusted accordingly so that the outflow streams, harvest and bleed, are equivalent to the inflow streams, basal medium and concentrated medium supplement. The medium that is fed to the bioreactor might vary between 100% basal medium and a ratio of 50:50 (v/v) of basal medium and concentrated medium supplement, whereby the ratio between basal medium and concentrated medium supplement is preferably between 99:1 and 60:40 (v/v), preferably between 98:2 and 80:20. By adding a proportion of the concentrated medium supplement the perfusion rate can be reduced by 10 to 80%, preferably by 15 to 40% of the perfusion rate when using only basal medium for maintaining the same VCD.

The basal medium and the concentrated medium supplement are preferably added either over the same medium inlet or over two different media inlets. If the concentrated medium supplement and the basal medium are added over the same medium inlet they are preferably blended in advance. The supply can be continuous, semi-continuous meaning continuous over a certain time frame or in portions.

Alternatively, it is possible to increase the overall perfusion rate during the whole perfusion process or during a certain time of the perfusion process by adding the concentrated medium supplement while keeping the perfusion rate of the basal medium constant or reducing the perfusion rate of the basal medium less compared to the addition of the concentrated medium supplement.

In another embodiment of the process of the present invention it is also possible to enlarge the VCD for a given perfusion rate by enlarging the proportion of concentrated medium supplement. That means the change in the overall medium composition by enlarging the proportion of the concentrated medium supplement results in an increase in VCD and a decrease in CSPR respectively. Also for this application, as for most perfusion processes, the process is preferably started by first running it in batch mode for some time while the number of cells, the VCD, increases. Typically, the process is run in batch mode for 2 to 8 days, preferably 3 to 5 days and then perfusion is started. In any case, perfusion is preferably turned on before the growth of the cells stops being exponential. This can for example be evaluated in prior cell line characterization experiments. A preferred cell density for starting perfusion is between 4 to 6 million cells per ml.

Perfusion is then turned on. As described above, perfusion can be started by feeding only basal cell culture medium or by feeding a combination of basal medium and medium concentrate. Preferably, the perfusion phase is started with only feeding basal cell culture medium. The perfusion rate is preferably around 0.5 to 3, preferably between 1 and 1.5. After reaching a constant VCD, the medium composition is then altered by increasing the proportion of the concentrated medium supplement. The perfusion rate is kept constant. This leads to a higher, stable cell density compared to a perfusion process with otherwise the same process parameters in which only the basal medium is applied.

It is also possible to combine two process variants by on the one hand adding a proportion of the concentrated medium supplement and on the other hand reducing the perfusion rate to a range in which the VCD is nevertheless higher compared to the same set-up with only basal medium. In this case the reduction of the perfusion rate is typically not as high as when maintaining the VCD but by adjusting the proportion of the concentrated medium supplement a person skilled in the art can adjust the perfusion rate and the VCD to optimal values whereby the perfusion rate is lower and the VCD is higher compared to a process in which only basal medium is fed to the cells.

Overall, the process is flexible. Perfusion rate can be kept constant, can be increased or decreased, either stepwise or continually, during perfusion state. The concepts can be applied to either steady state or dynamic perfusion approaches. The skilled person can adjust the scheme to the needs of the respective cell culture.

As can be seen from the above process variants, the addition of the concentrated medium supplement can be used to on the one hand make the process as effective as possible with higher productivity compared to a similar process with only basal medium. On the other hand it can be used to reduce volumetric medium consumption which is a known drawback in perfusion cell culture.

It has been found that the composition of the concentrated medium supplement is critical for reaching optimal results in optimizing the performance of the process and/or reducing medium consumption. The concentrated medium supplement preferably comprises at least three components, more preferably it comprises 5 to 30 components.

The concentrated medium supplement preferably comprises three or more amino acids and/or their equivalents.

Preferably it comprises three to 10 amino acids and/or their equivalents chosen from the group of asparagine, arginine, cysteine, glutamic acid, histidine, leucine, isoleucine, lysine, methionine, phenylalanine, threonine, tryptophan, tyrosine and valine.

Preferably, it comprises 2-oxoglutaric acid or equivalents thereof like the di-sodium salt.

Preferably, it comprises one or more vitamins.

It may also comprise least one saccharide.

In a very preferred embodiment, the concentrated medium supplement comprises phosphotyrosine as well as 2-oxoglutaric acid and/or sulphocysteine, especially(S)-2-Amino-3-(4-phosphonooxy-phenyl)-propionic acid sodium n salt, (S)-2-Amino-3-sulfosulfanyl-propionic acid sodium salt and/or 2-oxoglutaric acid disodium salt.

The concentrated medium supplement can be a dry powder to be dissolved in a liquid prior to use or a liquid medium. It is composed such that the pH in the liquid medium is between 6.5 and 10, preferably between pH 7 and 8.

The basal medium can be any cell culture medium suitable to maintain a perfusion cell culture. Suitable media are known to a person skilled in the art. Typically such basal media comprise one or more saccharide components, one or more amino acids, one or more vitamins or vitamin precursors, one or more salts, one or more buffer components, one or more co-factors and one or more nucleic acid components (nitrogenous bases) or their derivatives.

The present invention is also directed to a perfusion cell culture medium kit of parts comprising a basal cell culture medium and a concentrated medium supplement comprising at least phosphotyrosine.

In a preferred embodiment the concentration of the components of the concentrated medium supplement is at least 3 times, preferably 6 times the concentration of the equivalent component in the basal medium.

In a preferred embodiment the basal cell culture medium and the concentrated medium supplement are either liquid or in dry state for rehydration prior to use with a defined amount of liquid, most preferred they are granulated.

The present invention, without being limited thereby, is further illustrated by the following examples. All literature cited above or below are hereby incorporated by reference.

### EXAMPLES

The concentrated medium supplement, also called perfusion supplement or “PS”, can be used either for steady state perfusion or for dynamic perfusion.

### Steady State

### Example 1

For testing the use of the concentrated medium supplement in steady state perfusion, two stirred tank glass bioreactors with a volume of 1 L and a working volume of 0.89 L were used. Both were equipped with a pH probe, a DO probe, a temperature probe and a biomass probe and controlled with an Applikon MyControl system (bioreactor setpoints: pH=7.0+0.05; DO=40%; Temperature=37° C.±0.1° C., Agitation=300-500 rpm; Stirrer-type: marine impeller). An ATF device was used as cell retention device. Both bioreactors were filled with EX-CELL Advanced HD Perfusion® medium and inoculated with a cell density of 0.5×10⁶ viable cells/ml. The cell line used was a proprietary CHO-K1 cell line. Until day 5, the cells were grown in batch mode. Afterwards, perfusion was started at both bioreactors with a perfusion rate of 0.9 vvd with EX-CELL Advanced HD Perfusion® medium only.

On day 17, the perfusion rate for one of the bioreactors (condition “Perfusion Supplement”) was decreased to 0.8 vvd while blending 2% [v/v] perfusion supplement into the basal medium. The remaining condition (condition “Control”) was kept at 0.9 vvd. On day 26, the perfusion rate of the “Perfusion Supplement” bioreactor was decreased further to 0.6 vvd while increasing the concentration of the perfusion supplement in the basal medium to 6% [v/v].

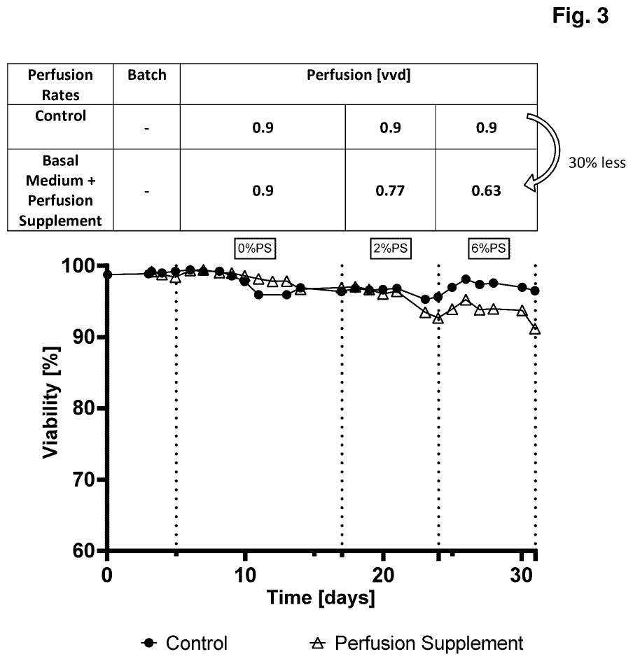

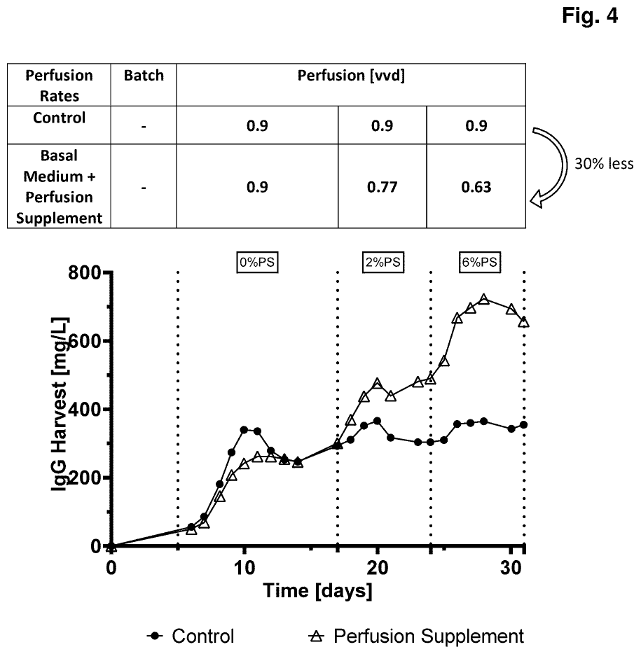

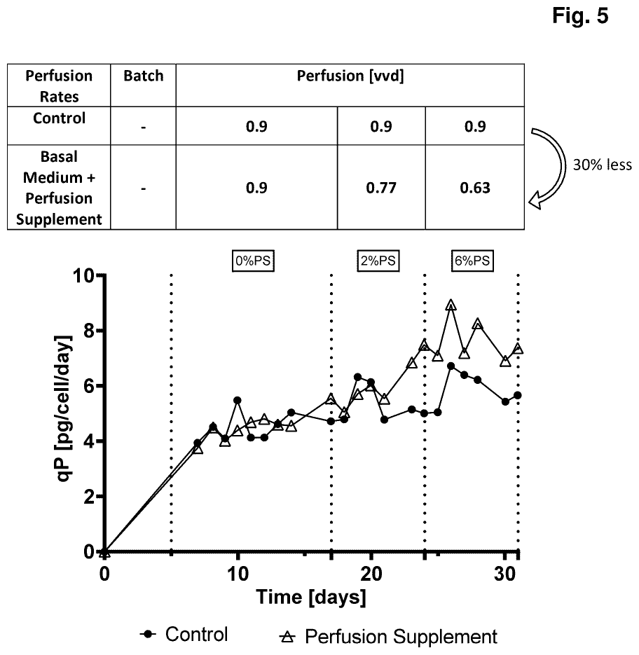

Viable cell density, viability, and IgG concentration were monitored throughout the run. Cells grew exponentially until 50×10⁶ viable cells/ml (FIG. 2). From then viable cell density was kept constant using a “bleed” realized through the bioreactor controller. Growth of both conditions was comparable. Viability (FIG. 3) of both conditions was comparable until day 21, decreased slightly for the “Perfusion Supplement” condition after decreasing the perfusion rate by 30% but maintained constant during this operational state. IgG concentrations (FIG. 4) were comparable for both conditions until day 17. When decreasing the perfusion rate to 0.77 vvd for the “Perfusion Supplement” condition, the IgG concentration increased through a higher residence time in the bioreactor. When decreasing the perfusion rate for the “Perfusion Supplement” condition even further to 0.63 vvd, the IgG concentration increased even further. The cell specific productivity (qP, calculated according to Bausch M, Schultheiss C, Sieck J B. Recommendations for Comparison of Productivity Between Fed-Batch and Perfusion Processes. Biotechnol J. 2019 February; 14 (2): e1700721), could be maintained throughout the run, or even increased after day 20 for the “Perfusion Supplement” condition (FIG. 5).

### Example 2

Instead of decreasing the perfusion rate stepwise, the perfusion supplement can be added alternatively during exponential growth to avoid the high perfusion rate necessary in steady state when using the basal medium only (compared to example 1). This example shows 2 options to obtain the same growth performance in steady state perfusion process. The basal medium in a perfusion set-up bioreactor is inoculated with 0.5×10⁶ viable cells/ml. Until day 3 cells are grown in batch mode. On day 3 perfusion is started with a perfusion rate of e. g. 0.2 and increased during growth of the cells. To obtain a stable steady state of 100×10⁶ viable cells/ml it is necessary to increase the perfusion rate of the “basal medium only” option according to prior art to 1 vvd to maintain the steady state. To decrease this medium demand, the perfusion supplement is already added during exponential growth (option 2 according to the present invention) allowing to reduce the overall final perfusion rate necessary to maintain the steady state by 30%. FIG. 6 shows an example with two options which lead to the same VCD.

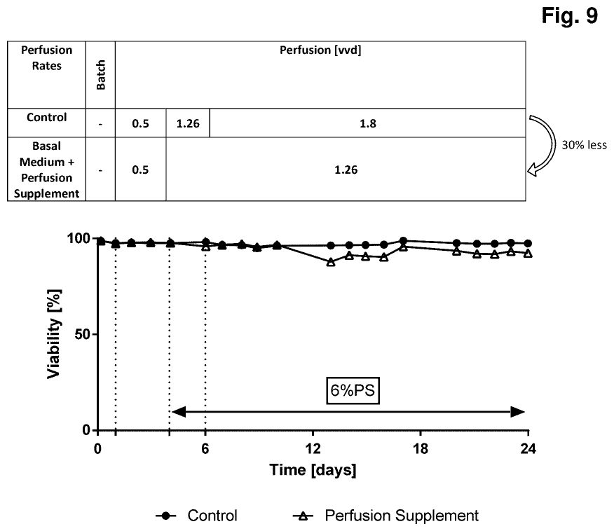

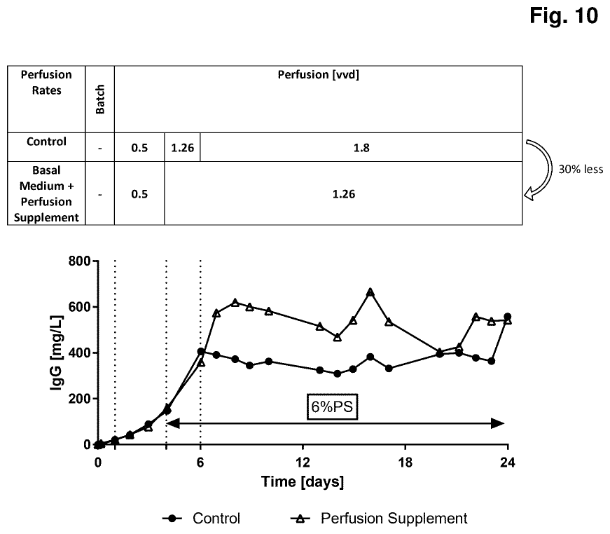

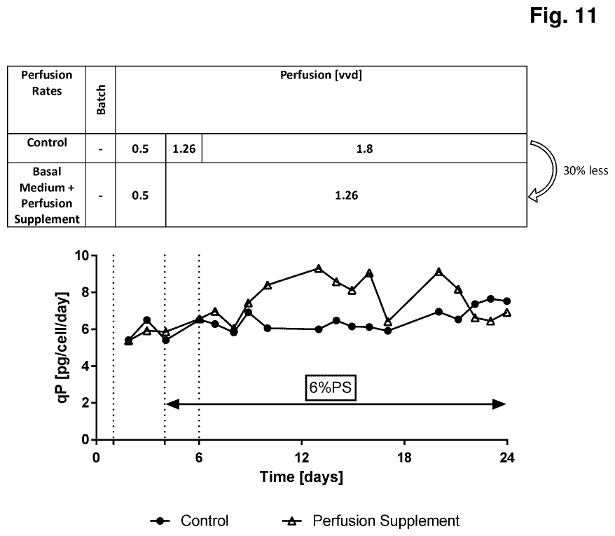

In addition, a perfusion process in two stirred tank glass bioreactors with a volume of 1 L and a working volume of 0.8 L was run (FIGS. 8-11). Both were equipped with a pH probe, a DO probe, a temperature probe and a biomass probe and controlled with an Applikon MyControl system (bioreactor setpoints: pH=6.90+0.05; DO=40%; Temperature=37.0° C.±0.1° C., Agitation=300-500 rpm; Stirrer-type: marine impeller). An ATF device was used as cell retention device. Both bioreactors were filled with EX-CELL Advanced HD Perfusion® medium and inoculated with a cell density of 2.2-2.4×10⁶ viable cells/ml. The cell line used was a proprietary CHO-K1 cell line. Until day 1, the cells were grown in batch mode. Afterwards, perfusion was started at both bioreactors with a perfusion rate of 0.5 vvd with EX-CELL Advanced HD Perfusion® medium only for both conditions. On day 4, the perfusion rate (still in growth phase) was increased to 1.26 vvd for both conditions while the basal medium for the condition “basal medium+perfusion supplement” was blended with 6% perfusion supplement. On day 6, the perfusion rate for the control (still in growth phase) was increased further to 1.8 vvd, while the condition “basal medium+perfusion supplement” was maintained at 1.26 vvd. Viable cell density, viability, and IgG concentration were monitored throughout the run. Cells grew exponentially until 100×10⁶ viable cells/ml (FIG. 8). From then viable cell density was kept constant using a “bleed” realized through the bioreactor controller. Growth of both conditions was comparable. Viability (FIG. 9) of both conditions was comparable until day 10 and decreased slighty for the perfusion supplement condition, however could be maintained above 90% until the end of the run. IgG concentration (FIG. 10) was lower for the control starting from day 6. Through calculation of the cell specific productivity (qP, FIG. 11) it can be seen that the increased titer for the perfusion supplement condition is not only an effect through the lower perfusion rate and the longer residence time of the product but also originates from a higher qP showing that the perfusion supplement can be used to decrease medium demand by 30% without compromising the process performance.

### Dynamic Perfusion (No Bleed)

### Example 3

The Perfusion supplement can also be used in dynamic perfusion. This example shows 2 options to obtain the same growth performance in dynamic perfusion. The basal medium in a perfusion set-up bioreactor is inoculated with 0.5×10⁶ viable cells/ml. Until day 3 cells are grown in batch mode. On day 3 perfusion is started with a perfusion rate of 1 vvd (option 1, state of the art). Cells grow exponentially first until a maximum viable cell density is reached. Afterwards cell density decreases. To obtain the same growth performance with a decreased medium demand, in option 2, the perfusion supplement is blended with the basal medium in a concentration between 2-10%, which decreases the perfusion rate in this example to 0.7 vvd without impacting the growth behavior. FIG. 7 shows the two options which lead to the same VCD.

1. A process for perfusion cell culture, comprising culturing cells in a bioreactor system comprising a bioreactor with a media inlet and a harvest outlet whereby
i. continuously or one or several times during the cell culture process new basal cell culture medium is inserted into the bioreactor via the media inlet
ii. continuously or one or several times during the cell culture process harvest is removed from the bioreactor via the harvest outlet
iii. one or several times during the cell culture process a concentrated medium supplement comprising at least phosphotyrosine is inserted into the bioreactor via the media inlet or an additional inlet.

2. A process according to claim 1, whereby the concentrated medium supplement is inserted at least 50% of the time, preferably at least 75% of the time of the cell culture without increasing the overall perfusion rate.

3. A process for perfusion cell culture according to claim 1, whereby the overall perfusion rate is reduced at least once during the course of the process.

4. A process for perfusion cell culture according to claim 1, whereby the concentrated medium supplement comprises at least five different components.

5. A process for perfusion cell culture according to claim 1, whereby the concentrated medium supplement comprises sulfocysteine and/or 2-oxoglutaric acid and/or salts thereof.

6. A process for perfusion cell culture according to claim 1, whereby the concentration of the components of the concentrated medium supplement is at least 3 times the concentration of the equivalent components in the basal medium.

7. A process for perfusion cell culture according to claim 1, whereby the overall perfusion rate (usually expressed in vvd) calculated over the duration of the process is at least 15-50% lower compared the lowest possible perfusion rate in the same process without using the concentrated medium supplement.

8. A process for perfusion cell culture according to claim 1, whereby the process is initiated by inoculating the bioreactor with cells and basal cell culture medium and performing batch cell culture until perfusion is started after 2 to 5 days while the cells are still in the exponential growth phase.

9. A process for perfusion cell culture according to claim 1, whereby concentrated medium supplement is added to the bioreactor continuously or one or several times after starting perfusion.

10. A process for perfusion cell culture according to claim 1, whereby the CSPR during perfusion is reduced stepwise or gradually through the addition of the concentrated medium supplement compared to a process which only uses basal medium.

11. A process for perfusion cell culture according to claim 1, whereby the CSPR before entering the perfusion state is reduced through the addition of the concentrated medium supplement compared to a process which only uses basal medium.

12. A process for perfusion cell culture according to claim 1, whereby the critical cell specific perfusion rate of this process is lower compared to an identical perfusion process in which no concentrated medium supplement is added but which otherwise has the same VCD.

13. A perfusion cell culture medium kit comprising a basal cell culture medium and a concentrated medium supplement comprising at least phosphotyrosine.

14. A perfusion cell culture medium kit according to claim 13, whereby the concentration of the components of the concentrated medium supplement is at least 3 times the concentration of the equivalent components in the basal medium.

15. A perfusion cell culture medium kit according to claim 13, whereby the concentrated medium supplement comprises one or more components selected from sulfocysteine and 2-oxoglutaric acid and salts thereof.

16. A perfusion cell culture medium kit according to claim 13, whereby the basal cell culture medium and the concentrated medium supplement are granulated.
